(19) United States
(12) Patent Application Publication
(10) Pub. No.: US 2016/0068420 A1
(43) Pub. Date: Mar. 10, 2016

(54) NOVEL RADIORESISTANT ALGA OF THE GENUS COCCOMYXA

(71) Applicants: COMMISSARIAT A L'ENERGIE ATOMIQUE ET AUX ENERGIES ALTERNATIVES, Paris (FR); INSTITUT MAX VON LAUE-PAUL LANGEVIN, Grenoble Cedex 9 (FR); CENTRE NATIONAL DE LA RECHERCHE SCIENTIFIQUE, Paris (FR)

(72) Inventors: Corinne Rivasseau, Cras (FR); Emmanuel Farhi, Cras (FR); Ariane Atteia, Marseille (FR); Danièle Pro, Gières (FR)

(73) Assignees: COMMISSARIAT A L'ENERGIE ATOMIQUE ET AUX ENERGIES ALTERNATIVES, Paris (FR); INSTITUT MAX VON LAUE-PAUL LANGEVIN, Grenoble Cedex 9 (FR); CENTRE NATIONAL DE LA RECHERCHE SCIENTIFIQUE, Paris (FR)

(21) Appl. No.: 14/786,324

(22) PCT Filed: Apr. 24, 2014
(86) PCT No.: PCT/IB2014/060984

§ 371 Date: Oct. 22, 2015

(30) Foreign Application Priority Data
Apr. 25, 2013 (FR) 13 53784

(51) Int. Cl. C02F 3/32; C12R 1/89; C12N 1/12

(52) U.S. Cl. CPC C02F 3/322; C12N 1/12; C12R 1/89; C02F 3/325; C02F 2101/006

(57) ABSTRACT

The invention relates to novel algae of the genus Coccomyxa, in particular the algae of a new species called C-IR3-4C, and their use for capturing metals from aqueous media, and in particular from radioactive media.

The invention relates to novel algae and to the use thereof for capturing metals from aqueous environments, and in particular from radioactive environments.

Radioactive effluents are produced mainly by nuclear power stations. They are principally water from spent fuel pools, water from decontamination tanks, water from nuclear plant cooling systems, or final effluents discharged into the environment, which ultimately contain radioactive compounds due to the activation of inactive compounds by radiation or to the release and dissolution of radioactive compounds. Other sources of radioactive effluents include nuclear medicine, hospitals that provide radiochemotherapy, and research laboratories that use radioactive materials. Effluents from certain nonnuclear industries (rare-earth mining, for example) are also concerned by the present invention.

Effluents, in particular water, containing radioactive compounds can be purified by a variety of physical and chemical methods. These methods, however, have high operating and equipment costs, require heavy maintenance and generate large volumes of radioactive waste. Moreover, their field of application is often limited. For example, ion-exchange resins are used to maintain low conductivity in water from nuclear plants. They become loaded with radioactive ions and, when saturated, are stored to await a suitable retreatment process or are stored under conditions employing toxic or highly reactive compounds.

Furthermore, some existing biological methods use bacteria, fungi, yeasts or plants, for example, to purify media (industrial effluents, natural media, etc.) contaminated with (radioactive or nonradioactive) toxic products. These methods use living organisms to concentrate, assimilate and reduce the toxicity (by modifying the chemical form) of polluting compounds, or use nonliving biomass or derivatives from living organisms to biosorb pollutants.

Biological methods generally have a broader field of application than physical and chemical methods. They do not require the addition of chemical reagents or products and generally make treatment less expensive, hence their economic advantage.

Plants in particular are good soil or water purifiers since they have an entire system of metabolites, proteins, enzymes, import mechanisms, membrane channels, internal structure, etc., which make them capable, as the case may be, of immobilizing toxic compounds, chelating them outside or inside the plant, incorporating them in varying amounts via specific or nonspecific import pathways, sequestering them inside cells, modifying their speciation so as to make them harmless or less toxic, making them less soluble, storing them in nontoxic form in the vacuole, etc.

Studies have shown that certain microorganisms are capable of concentrating via biosorption metal ions such as Ag, Al, Au, Co, Cd, Cu, Cr, Fe, Hg, Mn, Ni, Pb, Pd, Pt, U, Th, Zn, etc., in dilute solutions (White et al., International Biodeterioration & Biodegradation, 35: 17-40, 1995; U.S. Pat. No. 6,355,172). Biosorption is the capacity of biomass to bind heavy metals by means of metabolically-inactive physicochemical mechanisms via interactions with the functional groups of parietal compounds located at the surface of cells. For example, it has been disclosed that bacteria and mixtures of microorganisms can be used to nonselectively biosorb heavy metals (U.S. Pat. No. 7,479,220; PCT application WO 03/011487).

Other methods use dead biomass or derived compounds originating from the culture of living organisms to decontaminate metal-contaminated media. The methods employed include biologically-inactive physicochemical mechanisms, such as ion-exchange, for example with polysaccharides present in cell walls, complexation or adsorption.

Biomass derived from algae, for example from algal cell walls, has been used to purify metals contained in waste liquids (U.S. Pat. No. 4,769,223; PCT application WO 86/07346; U.S. Pat. No. 5,648,313; PCT application WO 2006/081932).

For treating media polluted with radioactive compounds, few methods call upon living organisms. Indeed, in the case of water contaminated with radioactive compounds or water located near radioactive sources, it is necessary to use radiotolerant or radioresistant organisms capable of withstanding the chemical and radiological toxicity of the contaminants and of binding these contaminants in a sufficient amount to be used in the context of an industrial decontamination process.

Methods for biological decontamination of radionuclides in nuclear effluents do not exist. However, the Fukushima nuclear accident on 11 Mar. 2011 led Japan to consider this type of solution with a material composed of a dehydrated microalga, Parachlorella sp. binos (WO 2010/024367). Five grams of this microalga could decontaminate 1 liter of water of a composition similar to water from the Fukushima reactors. The United States of America has long been interested in the feasibility of purifying radionuclides by means of biological methods. The use of Closterium moniliferum, an alga that incorporates strontium, was considered, but neither the ability to accumulate radioactive isotopes nor the radioresistance of this alga have been tested (Krejci M. R. et al., ChemSusChem, 4:470-473, 2011; Lovett R. A., Nature, doi:10.1038/news.2011.195, 2011).

In natural environments, organisms that accumulate radioactive compounds are generally subjected to low radioactivity. For example, immediately after the Chernobyl accident, with regard to aquatic environments, the dose rate of external ionizing radiation of the water in the reactor cooling tank did not exceed 100 μGy/h and the maximum cumulative dose over one year was 0.01 Gy in 1986. The dose rate originating from radionuclides deposited on sediments in the Pripyat River, located inside the 30 km zone around the power plant, increased at times to 0.4 mGy/h immediately after the accident (Kryshev and Sazykina, Journal of Environmental Radioactivity, 28: 91-103, 1995).

In most cases, the resistance to ionizing radiation of the microorganisms proposed for depolluting radioactivity-contaminated materials or effluents and/or for concentrating radioactive compounds has not been tested. Indeed, they are used to extract uranium (U) and thorium (Th), the activity of the main isotopes of which is low (for example, the activity of a solution containing 10 μg/1 of ²³⁸U or ²³⁵U is 0.13 or 0.8 Bq/l, respectively). U.S. Pat. No. 4,320,093 discloses the use of fungi of the genus Rhizopus for extracting uranium or thorium contained in aqueous effluents. Patent GB 1,472,626 discloses the use of unicellular green algae mutants obtained by X-ray irradiation of unicellular green algae prehabituated to uranium, and patent GB 1507003 discloses the use of various microorganisms, in particular the fungus Aspergillus niger and Oscillatoria cyanobacteria, for concentrating the uranium naturally present in sea water. U.S. Pat. No. 7,172,691 discloses the use of live photosynthetic algae of the genera Chlorella, Scenedesmus, Oocystis and Chlamydomonas for concentrating radioactive contaminants, in particular uranium, from aqueous media containing a uranium concentration of about 0-20 ppm, which represents an activity of 260 and 1600 Bq/1 for ²³⁸U and ²³⁵U, respectively. In comparison, the activity of water from spent nuclear fuel pools, which constitutes the living environment of the microalga of the invention, is about 300,000 Bq/1.

The most radioresistant organisms described to date are prokaryotes. The species Deinococcus radiodurans has an extraordinary capacity for resistance to ionizing radiation and grows under irradiation of 60 Gy/h and survives at doses of 15 kGy.

There is, however, no link between radiation tolerance and significant accumulation of radionuclides or metals. The use of the radioresistant bacterium Deinococcus radiodurans as a purifier of radioactive media requires the bacterium to be genetically modified in order to introduce genes enabling it to accumulate the metals of interest. For example, it has been proposed to use bacteria of the genus Deinococcus genetically modified to express enzymes capable of detoxifying or metabolizing organic compounds, metals or radionuclides, for purposes of in situ bioremediation of nuclear waste sites (PCT application WO 01/23526). More recently, bacteria of the species Kineococcus radiotolerans have been isolated and purified from a high-activity radioactive waste site. These bacteria have been described as being capable of degrading organic contaminants in the presence of ionizing radiation of a dose rate greater than 10 Gy/h, and their use for nonselectively depolluting radionuclides by means of biosorption has been proposed but not demonstrated (U.S. Pat. No. 7,160,715).

These two organisms have the disadvantage of being non-autotrophic bacteria, and therefore require an external supply of carbon nutrients in order to be able to employ their culture. In addition, their culture is more contamination-sensitive than that of autotrophic organisms, which need a less-rich, less bacterial contamination-sensitive culture medium.

Two radiotolerant, autotrophic organisms have been described. A microalga of the class Chlorophyceae, which tolerates ionizing radiation with an LD₅₀ of 6 kGy, has been studied (Farhi E. et al., J. Phys. Condens. Matt., 20: 104216, 2008). A novel microalga species, Coccomyxa actinabiotis, has also been identified, and it tolerates ionizing radiation with an LD₅₀ of 10 kGy (PCT application WO 2011/098979). Such radiation tolerance levels are rare, in particular in algae. In general, the algae have LD₅₀ values for resistance to ionizing radiation between 30 and 1200 Gy (International Atomic Energy Agency, IAEA, “Effects of ionizing radiation on aquatic organisms and ecosystems,” Technical Reports Series No. 172, 1976).

The alga Coccomyxa actinabiotis described in PCT application WO 2011/098979 is able to uptake and concentrate radioactive or nonradioactive metal ions in solution in aqueous medium and able to grow in radioactive medium.

For example, a test of purification of an actual-size pool containing this alga generated, with equivalent efficacy, a volume of waste about 1/100 that of the ion-exchange resins used in conventional methods for purifying water from nuclear plants (Rivasseau et al., Energy Environ. Sci., 6, 1230-1239, 2013).

The Inventors have now discovered that other microalgae of the genus Coccomyxa are able to uptake and concentrate radioactive ions in solution in aqueous medium and, moreover, are able to grow in radioactive medium.

They have discovered in particular, in spent nuclear fuel pools, a novel Coccomyxa species growing spontaneously in this environment rich in ionizing radiation and very poor in nutrients.

They have isolated and characterized this novel species, hereinafter referred to as Coccomyxa C-IR3-4C. Like Coccomyxa actinabiotis, this alga is both radiotolerant and an accumulator of radionuclides. This microalga was deposited according to the Budapest Treaty on 29 Jan. 2013 with the Culture Collection of Algae and Protozoa (CCAP, Scottish Association for Marine Science, Dunstaffnage Marine Laboratory, GB-Oban, Argyll, PA37 1QA, the United Kingdom) under number CCAP 216/26.

Algae of the species Coccomyxa C-IR3-4C are characterized in particular in that their genes corresponding to the 18S ribosomal RNA-ITS1-5.85 ribosomal RNA-ITS2-28S ribosomal RNA (start) contain a sequence having at least 96%, and, in order of increasing preference, at least 97%, 98%, or 99% identity with the sequence of SEQ ID NO: 1.

The percent identity indicated above is calculated after aligning the sequences, using the BLAST® tool, over a comparison window consisting of the entire sequence of SEQ ID NO: 1.

Algae of the species Coccomyxa C-IR3-4C can also be characterized in that the region corresponding to ITS1-5.85 rRNA-ITS2 has at least 96% identity with the corresponding region of the sequence of SEQ ID NO: 1. This threshold was estimated at 96% based on the observations of the Inventors, who show a maximum of 95% identity between this region in Coccomyxa C-IR3-4C and other Coccomyxae as well as a maximum of 88% identity between the ITS 1-5.8S rRNA-ITS2 of other Coccomyxae compared with one another.

The sequence of the Coccomyxa C-IR3-4C ribosomal RNA genes differs in particular from that of other species of the genus Coccomyxa by the nature of its ITS1 and ITS2 genes.

Coccomyxa C-IR3-4C can grow in radioactive medium, which enables it to uptake and metabolize radioactive compounds other than metals, in addition to being able to uptake and concentrate radioactive or nonradioactive metal ions in solution in radioactive or nonradioactive aqueous medium. Radioactive compounds other than metals include, for example, ³H or ¹⁴C. The latter can be metabolized in mineral form of CO₂, carbonate or hydrogen carbonate during photosynthesis or in organic form such as acetate in various algal metabolic processes.

The Inventors also discovered that another species of Coccomyxa, Coccomyxa chodatii (SAG strain no. 216-2 of the Culture Collection of Algae at Goettingen University), also radiotolerant, is able to concentrate radionuclides.

Consequently, the subject-matter of the present invention is the use of green algae of the genus Coccomyxa, and in particular the species Coccomyxa C-IR3-4C defined above and/or Coccomyxa chodatii, for capturing at least one radioactive or nonradioactive element selected from antimony (Sb) and the following metals: Cs, Ag, Co, Mn, Sr, Cu, Cr, Zn, Ni, Fe, actinides, such as uranium, lanthanides, and rare earths, such as Sc, Y, La, Ce, Pr, Nd, Pm, Sm, Eu, Gd, Tb, Dy, Ho, Er, Tm, Yb, Lu, and/or for capturing at least one of the radioisotopes ¹⁴C and ³H, from an aqueous medium containing said element and/or said radioisotope in solution.

More particularly, the subject-matter of the present invention is a method for capturing at least one radioactive or nonradioactive element selected from Cs, Ag, Co, Mn, Sr, Cu, Cr, Zn, Ni, Fe, Sb, actinides, lanthanides and rare earths, and/or at least one of the radioisotopes ¹⁴C and ³H, from an aqueous medium containing said element and/or said radioisotope in solution, characterized in that said capturing is carried out by incubating in said aqueous medium green algae of the genus Coccomyxa, in particular Coccomyxa C-IR3-4C and/or Coccomyxa chodatii.

Advantageously, said element is a metal selected from Ag, Co, Cs, U, Mn, Cu and Sr, and rare earths.

According to a particular embodiment, the invention relates to a method for capturing at least one radioactive or nonradioactive element selected from Sr and Cu from an aqueous medium containing said element in solution, characterized in that said capturing is carried out by incubating, in said aqueous medium, unicellular green algae of the genus Coccomyxa.

According to a preferred embodiment of the present invention, said aqueous medium is radioactive medium, i.e., medium in which said algae are subjected to a dose rate ranging from several μGy/h up to several kGy/h. According to a preferred arrangement of this embodiment, the element to be captured is a metal selected from those indicated above, in the form of a stable or radioactive isotope, or in the form of a mixture of isotopes.

According to another preferred embodiment of the present invention, said aqueous medium is nonradioactive medium. The element to be captured is preferably a metal selected from rare earths.

According to another embodiment of the present invention, said aqueous medium is acidic medium. This is particularly advantageous, because the solutions used to dissolve urban waste are generally highly acidic (pH 1 or pH 2). The inventors have shown (Example 7.4) that the unicellular green algae Coccomyxa CCAP 216/26 were able to survive and to uptake rare earths at pH 1. The element to be captured is preferably a metal selected from rare earths. Unicellular green algae of the genus Coccomyxa can thus be advantageously used, in the context of the present invention, for depolluting acidic aqueous medium of pH 1, of pH between 1 and 2, of pH between 2 and 3, or of pH between 3 and 6.

The incubation time of the algae in the aqueous medium can vary, in particular according to, firstly, the element(s) concerned and, secondly, the nature of the aqueous medium from which the capturing must be carried out. It will generally be at least 1 hour, and range up to several months, or even several years. For example, if it is desired to capture Ag or Cs or U, an incubation time of about 24 hours could be sufficient to capture the major part thereof.

The maximum incubation time that can be envisaged will in fact depend mainly on the capacity for growth and survival on the algae in the aqueous medium.

In the presence of light and carbon dioxide (introduced by contact with ambient air, agitation of the cultures or bubbling), algae of the genus Coccomyxa, and in particular of the species Coccomyxa C-IR3-4C and Coccomyxa chodatii, can grow and live for very long periods of time in weakly mineralized water (conductivity 1 to 2 μS/cm), at a pH of 6 to 7 and a temperature of 20 to 30° C. Since these green algae need light to carry out photosynthesis and to produce their organic matter, their growth stops when they are placed in the dark.

Thus, according to an embodiment of the method in accordance with the present invention, the growth of the green algae of the species Coccomyxa C-IR3-4C and Coccomyxa chodatii can be controlled by regulating the illumination of the aqueous medium comprising said algae.

Coccomyxa C-IR3-4C algae can also grow and live for several years in weakly radioactive medium, where they are subjected to radiation of less than or equal to 0.1 mGy/h.

For implementing the method in accordance with the invention, the algae can be used in suspension in the aqueous medium from which the capture must be carried out, with agitation in order to prevent said algae from agglomerating. They can also be bound on a solid, smooth, porous support or on beads, placed in said aqueous medium.

The method can be transported, i.e., the algae are brought into contact with the medium to be depolluted in an enclosure separate from the nuclear plant, or are implemented in situ; in the latter case the algae are then implanted directly in the medium to be depolluted.

In the context of present the invention, the terms “depollution” and “decontamination” are synonymous and can be employed interchangeably for radioactive or nonradioactive elements. In the case of an in situ method of capture or decontamination, the algae can remain in the plant as long as they do not interfere with the plant's operations. By way of indication, their growth can be controlled by the intensity of illumination (darkness or weak illumination), or the choice of the wavelength of the lamps (for example, yellow-green inactinic light). In addition, the water can be filtered so as to control algal growth by capturing algae suspended therein. If it proves necessary to remove the algae, they can be completely destroyed by means of oxidation, for example using 4 g/l hydrogen peroxide for 1 to 7 days. This operation is accompanied by a release of metals, and thus should preferably be carried out section by section in the plant, with recovery of the effluents containing a concentration of metals.

In the case of a transported method or an in situ method, at the end of incubation, the algae having captured metals are collected by conventional means (filtration, decanting, centrifugation, etc.). They can then be discarded as waste, optionally after being dried and/or burned, without prior extraction of the metals contained therein.

Advantageously, the elements captured by the algae can be recovered in order to recycle said elements. This recovery can be carried out by any suitable means.

In particular, the elements can be recovered after destruction of the algae. This destruction can, for example, be carried out by lysis of the algae, advantageously by oxidation, for example with 4 g/l hydrogen peroxide for 1 to 7 days. It can also be carried out by incinerating the algae.

The elements can be recovered by adding a complexing agent such as ethylenediaminetetraacetic acid (EDTA) or by adding acid.

The method in accordance with the invention can be implemented in all cases where it is desired to extract radioactive or nonradioactive elements, and in particular those mentioned above, from an aqueous medium, for purposes of mining operations, or of depollution of aqueous effluents, in particular of radioactive effluents.

In addition to their strong capacity for specifically capturing and concentrating the elements mentioned above (Cs, Ag, Co, Mn, Sr, Cu, Cr, Zn, Ni, Fe, Sb, actinides, lanthanides and rare earths, such as Sc, Y, La, Ce, Pr, Nd, Pm, Sm, Eu, Gd, Tb, Dy, Ho, Er, Tm, Yb, Lu, whether they are radioactive or nonradioactive), algae of the genus Coccomyxa, and in particular of the species Coccomyxa C-IR3-4C and Coccomyxa chodatii, also have properties of nonspecific uptake of other elements, in particular metals, notably due to their extracellular mucilage consisting of polysaccharides, which have the property of complexing cations.

The method in accordance with the invention is therefore particularly suitable for depolluting and for decontaminating aqueous media or soils (peat bogs, marshes) contaminated with radioactive or nonradioactive metals, and more particularly radioactive media, such as water from storage pools or light water from secondary cooling systems of nuclear power stations or reactors, or effluents from nuclear power stations discharged into the environment, or effluents from hospital facilities.

The present invention can be implemented not only with unicellular green algae of the species Coccomyxa C-IR3-4C, or Coccomyxa chodatii, or another alga belonging to the genus Coccomyxa, or mixtures thereof, but also with a mixture of microorganisms comprising at least one unicellular green alga belonging to the genus Coccomyxa, in particular a microalga of the species Coccomyxa C-IR3-4C or the species Coccomyxa chodatii, and at least one microorganism, in particular a bacterium, a fungus, a yeast, another unicellular alga, and/or a multicellular plant, preferably radioresistant or radiotolerant, capable of concentrating metal ions in solution and/or of capturing and metabolizing radioactive compounds other than metals (for example, ³H or ¹⁴C). The multicellular microorganisms and plants that can be used in combination with the unicellular green algae of the genus Coccomyxa are in particular those cited above, in particular those that are radioresistant or radiotolerant. In the case where the present invention is applied to a radioactive aqueous medium, the incubation time of the mixture of microorganisms will depend on the individual resistance of the microorganisms making up the mixture. Similarly, the culture conditions may be adjusted in order to promote the growth of one or more microorganisms making up the mixture.

The present invention will be understood more clearly from the further description which follows, which refers to examples illustrating the isolation and the characterization of the species Coccomyxa C-IR3-4C, the use thereof for decontaminating a radioactive aqueous medium, and the characterization of the radionuclide uptake properties of Coccomyxa chodatii.

### EXAMPLE 1

### Isolation and Characterization of Coccomyxa C-IR3-4C

The microalga was collected from a spent nuclear fuel pool. The water contained in this pool has a pH between 6 and 7, conductivity of 1 to 2 μS/cm, is in contact with ambient air and contains dissolved radioactive elements. Its temperature varies between 22 and 28° C. and is on average 25° C. The radiological activity in the pool is highly variable from weak to very strong, depending on the measuring points.

The presence of films of green organic matter was observed on the walls and various surfaces of this pool. Samples were taken, which when observed under a microscope showed that this was a unicellular green microalga.

### Culture Conditions

The samples were stored and cultured in light, on nutrient agar, in a sterile environment, at a temperature of 20 to 23° C. The nutrient medium is Bold's Basal Medium (BBM, Sigma), pure or diluted in demineralized water. BBM is traditionally used to culture green algae. The culture medium has a pH of 6.4. Its composition is indicated in Table I below.

TABLE I

| Components in g/l | BBM |
|---|---|
| NaNO3 | 0.25 |
| KH2PO4 | 0.175 |
| K2HPO4 | 0.075 |
| MgSO4, 7H2O | 0.075 |
| FeSO4, 7H2O | 0.005 |
| CaCl2, 2H2O | 0.025 |
| NaCl | 0.025 |
| Na2EDTA | 0.01 |
| KOH | 0.006 |
| H3BO4 | 12.86 |
| MnCl2, 4H2O | 1.81 |
| ZnSO4, 7H2O | 0.222 |
| Na2MoO4, 2H2O | 0.39 |
| CuSO4, 5H2O | 0.079 |
| Co(NO3)2, 6H2O | 0.049 |

The algae were placed in culture on solid BBM agar. Colonies were isolated and then diluted in order to spread the isolated cells over agar culture medium. This operation was repeated five times in order to obtain Coccomyxa strain C-IR3-4C, which was sequenced.

A sample of this culture was deposited according to the Budapest Treaty on 29 Jan. 2013 with the Culture Collection of Algae and Protozoa (CCAP), under number CCAP 216/26.

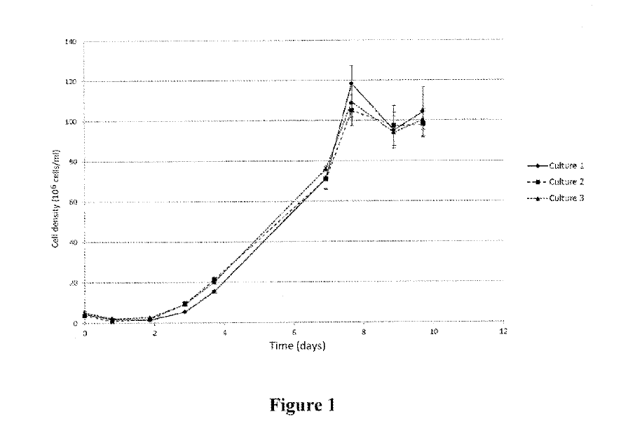

In liquid BBM, the Coccomyxa strain C-IR3-4C microalgae increased with an exponential growth phase. Microalgal growth was measured by counting the cell density of the algal population over time on three samples of algae cultured in BBM diluted 1:2, in an Infors Multitron incubator maintained at 21±1° C., with 100 rpm shaking and 90±10 PAR continuous illumination. The count is made using a Malassez counting chamber under a microscope (X40 objective magnification). The growth curves are presented in FIG. 1, which shows the change in the cell density of the algal population as a function of culture time.

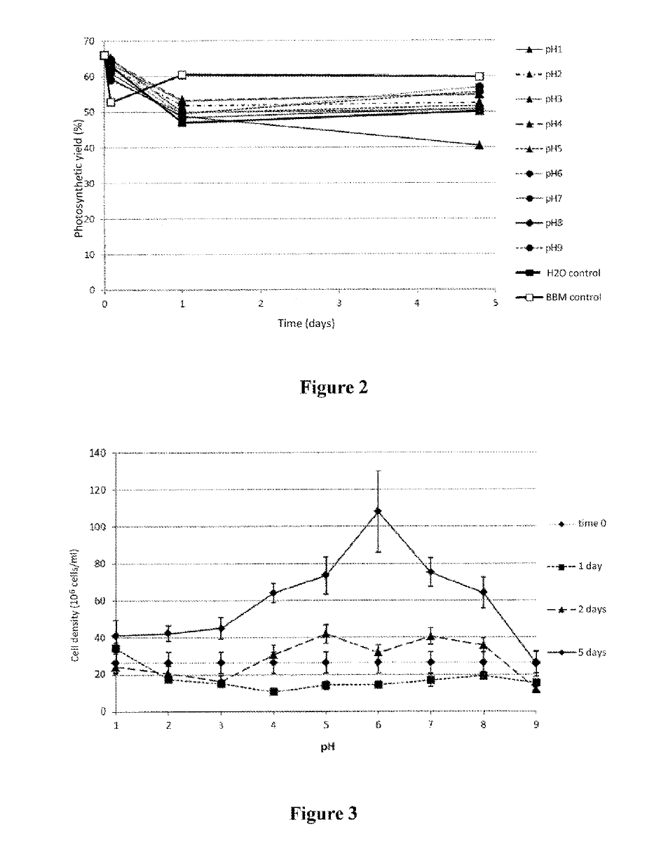

Coccomyxa strain C-IR3-4C microalgae are capable of living in liquid medium over a wide range of pH. The algae were cultivated in demineralized water the pH of which was adjusted to the target values by adding HCl or KOH. The cultures are grown in an Infors Multitron incubator maintained at 21±1° C., with 100 rpm shaking and 90±10 PAR continuous illumination. The pH of the media is checked daily and readjusted as needed. The state of the algae as a function of the pH of the medium is evaluated by their photosynthetic yield, which is an indicator of the overall physiological state of the cells. Photosynthetic yield is measured using a PAM 103 fluorometer. FIG. 2 shows the photosynthetic yield values of the microalgae as a function of different pH values. It shows that they can live in media of pH 1 to pH 9 (pH range tested). FIG. 2 shows that the microalgae retain a good physiological state in medium composed of demineralized water and that they retain a good physiological state whatever the pH.

Coccomyxa strain C-IR3-4C microalgae are also capable of growing in liquid medium over a wide range of pH. FIG. 3 shows the change in the cell density of the algal population as a function of pH over time in media of pH 1 to pH 9. The culture conditions are identical to those of FIG. 2. The cell density count is made using a Malassez counting chamber under a microscope (×40 magnification). To obtain rapid growth and high microalgae density, a pH range of 4 to 8 will be used.

### Morphological and Biochemical Features

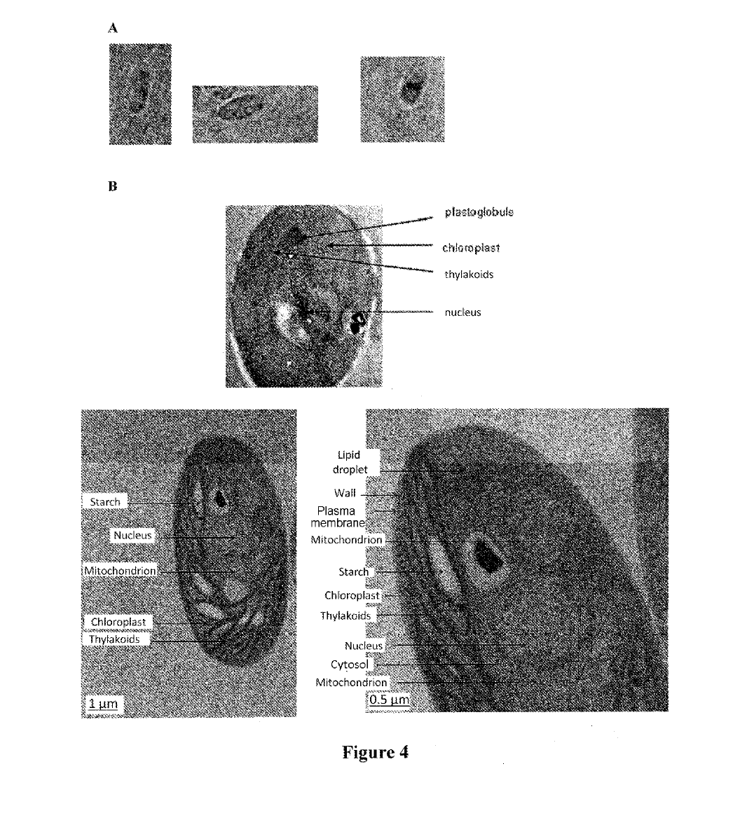

The isolated microalgae observed by photon microscopy and electron microscopy are unicellular, ellipsoidal and nucleated (FIGS. 4A and 4B). FIGS. 4A and 4B are photographs of these microalgae observed, respectively, by photon microscopy (Zeiss Axioplan 2 binocular microscope, 1300 magnification) and by transmission electron microscopy (JEOL 1200EX and Philips CM 120). The observations with the Philips CM 120 TEM were carried out at the Technological Center for Microstructures—Claude Bernard University Lyon 1). The microalgae contain a chloroplast (perhaps several) which contains chlorophyll and which is the site of photosynthesis. Other organelles, in particular the vacuole, occupy the rest of the cell.

The UV-visible absorption spectrum of this organism shows the presence of chlorophyll a (absorption peak at 663 nm), chlorophyll b (absorption peak at 647 nm) and carotenoids (absorption peak at 470 nm).

### Amplification and Sequencing of Ribosomal DNA Genes

Total DNA of the C-IR3-4C microalga isolated as described above was extracted using the Wizard Genomic DNA Purification Kit (Promega).

The region of the genome covering the 18S rRNA-ITS1-5.8S rRNA-ITS2-28S rRNA (the first 500 bases) ribosomal DNA genes was amplified by PCR.

The primers used are EAF3: TCGACAATCTGGTTG ATCCTGCCAG (SEQ ID NO: 2) and ITS055R: CTCCTTGGTC CGTGTTTCAAGACGGG (SEQ ID NO: 3), traditionally used to amplify microalgal rRNA genes.

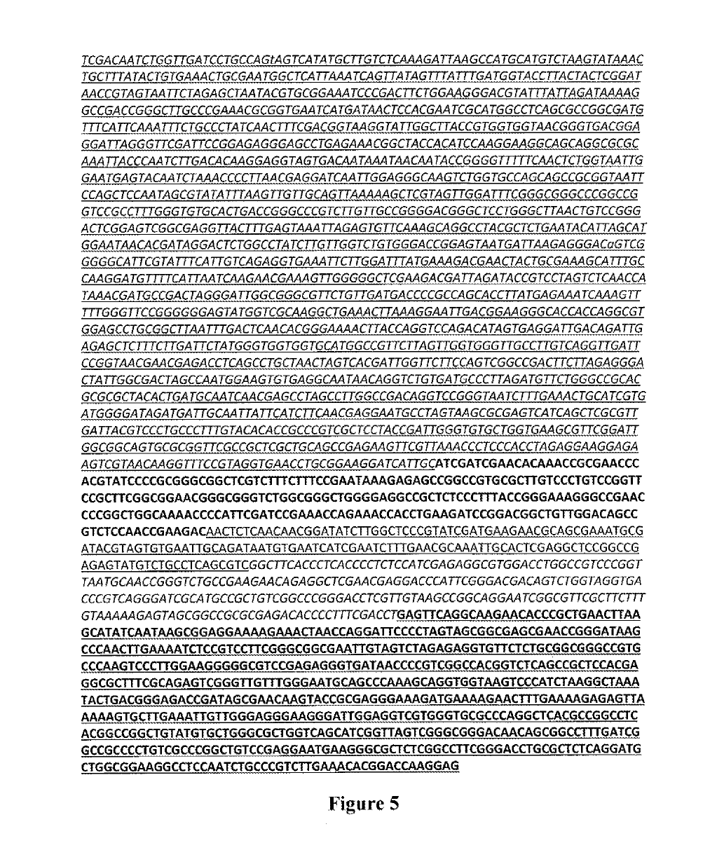

The amplification products obtained by using DNA isolated from two independent cultures were sequenced and are 3101 bases. The sequence of these amplification products is shown in FIG. 5 and in the sequence listing in the appendix under number SEQ ID NO: 1. This sequence represents the sequence of the genomic region covering the 18S rRNA-ITS1-5.8S rRNA-ITS2-28S rRNA (start) ribosomal DNA genes of the alga Coccomyxa C-IR3-4C. The portion corresponding to the 18S rRNA is underlined and italicized, the ITS1 is in bold, the 5.8S rRNA is underlined, the ITS2 is italicized, and the 28S rRNA is in bold and underlined.

The BLASTN algorithm (Altschul et al., Nucleic Acids Research, 25: 3389-3402, 1997) was used to search databases for ribosomal RNA gene sequences having maximum identity with the sequence of SEQ ID NO: 1. This search revealed that the species characterized as the most similar to microalga C-IR3-4C belong to the genus Coccomyxa.

The comparison of the sequences corresponding to the RNA of the small ribosomal subunit (18S rRNA) of microalga C-IR3-4C (1807 bp) and of other Coccomyxa species listed in the databases was carried out by multiple sequence alignment using the BLAST® algorithm. Table II below presents the results of this sequence comparison. The sequences of the other species can be accessed in the GenBank database, and the corresponding accession numbers are also indicated in Table II.

TABLE II

| | | Total | Query | Max |
|---|---|---|---|---|
| Accession | Description | score | coverage | ident |
| HQ317304.1 | Coccomyxa rayssiae isolate UTEX273 small | 3290 | 99% | 99% |
| | subunit ribosomal RNA gene, partial | | | |
| | sequence | | | |
| FN298927.1 | Coccomyxa sp. CCAP 216/24 18S rRNA | 3269 | 98% | 99% |
| | gene (partial), ITS1, 5.8S rRNA gene, ITS2 | | | |
| | and 28S rRNA gene (partial), strain CCAP | | | |
| | 216/24 | | | |
| FN298926.1 | Pseudococcomyxa simplex 18S rRNA gene | 3264 | 98% | 99% |
| | (partial), ITS1, 5.8S rRNA gene, ITS2 and | | | |
| | 28S rRNA gene (partial), strain SAG 216-9a | | | |
| HE586518.1 | Choricystis sp. GSE4G genomic DNA | 3262 | 98% | 99% |
| | containing 18S rRNA gene, ITS1, | | | |
| | 5.8S rRNA gene and ITS2, strain GSE4G | | | |
| FR865679.1 | ‘Chlorella’ saccharophila genomic DNA | 3262 | 98% | 99% |
| | containing 18S rRNA gene, ITS1, culture | | | |
| | collection CCAP 211/60 | | | |
| FN597598.1 | Coccomyxa chodatii SAG 216-2 | 3254 | 98% | 99% |
| FJ946891.1 | Trebouxiophyceae sp. VPL5-6 18S | 3234 | 97% | 99% |
| | ribosomal RNA gene, partial sequence | | | |
| FJ648514.1 | Pseudococcomyxa simplex strain UTEX 274 | 3229 | 97% | 99% |
| | 18S ribosomal RNA gene, complete | | | |
| | sequence | | | |
| FN597599.1 | Coccomyxa peltigerae SAG 216-5 | 3221 | 98% | 99% |
| HE586504.1 | Pseudococcomyxa simplex genomic DNA | 3195 | 96% | 99% |
| | containing 18S rRNA gene, ITS1, | | | |
| | 5.8S rRNA gene and ITS2, strain CAUP H | | | |
| | 102 | | | |
| HE586513.1 | Coccomyxa sp. KN-2011-E4 genomic DNA | 3181 | 96% | 99% |
| | containing 18S rRNA gene, ITS1, | | | |
| | 5.8S rRNA gene and ITS2, strain E4 | | | |
| FN298928.1 | Coccomyxa sp. CCAP 211/97 18S rRNA | 3181 | 98% | 99% |
| | gene (partial), ITS1, 5.8S rRNA gene, ITS2 | | | |
| | and 28S rRNA gene (partial), strain CCAP | | | |
| | 211/97 | | | |
| AB742451.1 | Coccomyxa sp. KGU-D001 gene for 18S | 3158 | 95% | 99% |
| | ribosomal RNA, partial sequence | | | |
| HE586512.1 | Coccomyxa sp. KN-2011-C15 genomic DNA | 3129 | 97% | 99% |
| | containing 18S rRNA gene, strain C15 | | | |
| FR850476.1 | Coccomyxa actinabiotis 18S rRNA, ITS1, | 3126 | 98% | 99% |
| | 5.8S rRNA, ITS2 (CCAP 216-25) | | | |
| HE586505.1 | Pseudococcomyxa simplex genomic DNA | 3109 | 96% | 99% |
| | containing 18S rRNA gene, strain CAUP H | | | |
| | 103 | | | |
| HE586514.1 | Coccomyxa sp. KN-2011-T2 genomic DNA | 3103 | 97% | 98% |
| | containing 18S rRNA gene, ITS1, | | | |
| | 5.8S rRNA gene and ITS2, strain T2 | | | |
| AY422078.1 | Paradoxia multiseta 18S small subunit | 3081 | 93% | 99% |
| | ribosomal RNA gene, partial sequence | | | |
| AM981206.1 | Coccomyxa sp. CPCC 508 18S rRNA gene, | 3077 | 99% | 98% |
| | strain CPCC 508 | | | |
| AJ302939.1 | Coccomyxa sp. SAG 2325 18S rRNA gene, | 3070 | 99% | 98% |
| | culture collection SAG:2325 | | | |
| AM167525.1 | Coccomyxa glaronensis 18S rRNA gene, | 3061 | 99% | 97% |
| | strain CCALA 306 | | | |
| HE586507.1 | Ellipsoidion sp. UTEX B SNO113 genomic | 3059 | 98% | 98% |
| | DNA containing 18S rRNA gene, strain | | | |
| | UTEX B SNO113 | | | |
| HE586519.1 | Monodus sp. CR2-4 genomic DNA | 3055 | 98% | 98% |
| | containing 18S rRNA gene, ITS1, | | | |
| | 5.8S rRNA gene and ITS2, strain CR2-4 | | | |
| FR865588.1 | Chlamydomonas bipapillata genomic DNA | 3053 | 98% | 98% |
| | containing 18S rRNA gene, ITS1, culture | | | |
| | collection CCAP 11/47 | | | |
| EU282454.1 | Paradoxia sp. 294-GA206 18S ribosomal | 3044 | 93% | 99% |
| | RNA gene, partial sequence | | | |
| GQ122371.1 | Trebouxiophyceae sp. KMMCC FC-10 18S | 3042 | 92% | 99% |
| | ribosomal RNA gene, partial sequence | | | |
| HE586506.1 | Monodus sp. UTEX B SNO83 genomic DNA | 3035 | 98% | 97% |
| | containing 18S rRNA gene, ITS1 and | | | |
| | 5.8S rRNA gene, strain UTEX B SNO83 | | | |
| HE586509.1 | Coccomyxa sp. KN-2011-C10 genomic DNA | 3022 | 97% | 98% |
| | containing 18S rRNA gene, strain C10 | | | |
| JQ946088.1 | Coccomyxa sp. XDL-2012 18S ribosomal | 3014 | 92% | 99% |
| | RNA gene, partial sequence | | | |
| EU127471.1 | Coccomyxa sp. Flensburg fjord 2 18S | 3014 | 98% | 97% |
| | ribosomal RNA gene, partial sequence | | | |
| FJ648513.1 | Coccomyxa mucigena strain SAG 216-4 | 2998 | 97% | 97% |
| | 18S ribosomal RNA gene, complete | | | |
| | sequence | | | |
| HE586508.1 | Coccomyxa sp. KN-2011-C4 genomic DNA | 2964 | 96% | 97% |
| | containing 18S rRNA gene, ITS1, | | | |
| | 5.8S rRNA gene and ITS2, strain C4 | | | |
| EU127470.1 | Coccomyxa sp. Flensburg fjord 1 18S | 2953 | 97% | 97% |
| | ribosomal RNA gene, partial sequence | | | |
| AY195970.1 | Choricystis sp. AS 5-1 18S ribosomal RNA | 2931 | 99% | 96% |
| | gene, partial sequence | | | |
| AY197620.1 | Chlorella sp. Mary 9/21 BT-10w 18S | 2913 | 99% | 96% |
| | ribosomal RNA gene, partial sequence | | | |
| AB080308.1 | Chlorella vulgaris gene for 18S rRNA, partial | 2896 | 99% | 96% |
| | sequence | | | |
| EF106784.1 | Chlamydomonas sp. CCMP 681 | 2235 | 86% | 93% |

This sequence comparison shows that the species the most similar to strain C-IR3-4C are Coccomyxa rayssiae strain UTEX273, Coccomyxa strain CCAP 216/24, Pseudococcomyxa simplex strain SAG 216-9a, Coccomyxa chodatii strain SAG 216-2, Pseudococcomyxa simplex strain UTEX 274, Coccomyxa peltigerae strain SAG 216-5, Pseudococcomyxa simplex strain CAUP H 102, Coccomyxa strain KN-2011-E4, Coccomyxa strain CCAP 211/97, as well as other strains belonging to the genus of Coccomyxae with 99% sequence identity, then other strains still belonging to the genus of Coccomyxae such as Coccomyxa strain CPCC 508 or Coccomyxa strain SAG 2325 with 98% sequence identity, then other Coccomyxae such as Coccomyxa glaronensis strain CCALA 306, Coccomyxa sp. Flensburg fjord 2 or Coccomyxa mucigena strain SAG 216-4 with 97% sequence identity.

These high identity scores obtained for Coccomyxa strain C-IR3-4C compared with the genus Coccomyxa are close to those obtained after comparison of the sequences of Coccomyxae between one another (96-99%) and far from the score obtained for the sequence comparison with a unicellular microalga belonging to another genus (Chlamydomonas sp. CCMP681 (EF106784.1), 93% identity). This indicates that strain CCAP 216/26 is a member of the genus Coccomyxa.

Furthermore, the comparison of the ITS region sequences of strain C-IR3-4C with those of other Coccomyxae was also performed. Table III below presents the results of this sequence comparison of the ITS1-5.85 rRNA-ITS2 regions. The sequences of the other species can be accessed in the GenBank database, and the corresponding accession numbers are also indicated in Table III.

TABLE III

| | | Total | Query | Max |
|---|---|---|---|---|
| Accession | Description | score | coverage | ident |
| AY293967.1 | Coccomyxa solarinae var. saccatae internal | 1035 | 99% | 95% |
| | transcribed spacer 1, 5.8S ribosomal RNA | | | |
| | gene, and internal transcribed spacer 2, | | | |
| | complete sequence | | | |
| AY293966.1 | Coccomyxa solarinae var. bisporae internal | 1030 | 99% | 95% |
| | transcribed spacer 1, 5.8S ribosomal RNA | | | |
| | gene, and internal transcribed spacer 2, | | | |
| | complete sequence | | | |
| AY293965.1 | Coccomyxa solarinae var. croceae internal | 1009 | 99% | 94% |
| | transcribed spacer 1, 5.8S ribosomal RNA | | | |
| | gene, and internal transcribed spacer 2, | | | |
| | complete sequence | | | |
| HE586513.1 | Coccomyxa sp. KN-2011-E4 genomic DNA | 1002 | 100% | 95% |
| | containing 18S rRNA gene, ITS1, 5.8S rRNA | | | |
| | gene and ITS2, strain E4 | | | |
| HE586545.1 | Coccomyxa sp. KN-2011-E5 genomic DNA | 985 | 99% | 94% |
| | containing 18S rRNA gene, ITS1, 5.8S rRNA | | | |
| | gene and ITS2, strain E5 | | | |
| HE586551.1 | Coccomyxa sp. KN-2011-T5 genomic DNA | 939 | 93% | 94% |
| | containing ITS1, 5.8S rRNA gene and ITS2, | | | |
| | strain T5 | | | |
| AY293964.1 | Coccomyxa peltigerae var. variolosae | 910 | 94% | 94% |
| | internal transcribed spacer 1, 5.8S ribosomal | | | |
| | RNA gene, and internal transcribed spacer | | | |
| | 2, complete sequence | | | |
| HQ335215.1 | Coccomyxa sp. S2 internal transcribed | 745 | 83% | 92% |
| | spacer 1, partial sequence; 5.8S ribosomal | | | |
| | RNA gene, complete sequence; and internal | | | |
| | transcribed spacer 2, partial sequence | | | |
| HQ335216.1 | Coccomyxa sp. S10 internal transcribed | 745 | 83% | 92% |
| | spacer 1, partial sequence; 5.8S ribosomal | | | |
| | RNA gene, complete sequence; and internal | | | |
| | transcribed spacer 2, partial sequence | | | |
| FN298926.1 | Pseudococcomyxa simplex 18S rRNA gene | 673 | 92% | 95% |
| | (partial), ITS1, 5.8S rRNA gene, ITS2 and | | | |
| | 28S rRNA gene (partial), strain SAG 216-9a | | | |
| HE586504.1 | Pseudococcomyxa simplex genomic DNA | 668 | 92% | 94% |
| | containing 18S rRNA gene, ITS1, 5.8S rRNA | | | |
| | gene and ITS2, strain CAUP H 102 | | | |
| FN298927.1 | Coccomyxa sp. CCAP 216/24 18S rRNA | 651 | 100% | 88% |
| | gene (partial), ITS1, 5.8S rRNA gene, ITS2 | | | |
| | and 28S rRNA gene (partial), strain CCAP | | | |
| | 216/24 | | | |
| AY328524.1 | Coccomyxa rayssiae strain SAG 216-8 18S | 640 | 92% | 94% |
| | ribosomal RNA gene, partial sequence; | | | |
| | internal transcribed spacer 1, 5.8S ribosomal | | | |
| | RNA gene and internal transcribed spacer 2, | | | |
| | complete sequence; and 26S ribosomal | | | |
| | RNA gene, partial sequence | | | |
| HE586526.1 | Pseudococcomyxa sp. KN-2011-A1 genomic | 568 | 90% | 91% |
| | DNA containing ITS1, 5.8S rRNA gene and | | | |
| | ITS2, intragenomic variability copy B, strain | | | |
| | A1 | | | |
| HE586525.1 | Pseudococcomyxa sp. KN-2011-A1 genomic | 566 | 90% | 91% |
| | DNA containing ITS1, 5.8S rRNA gene and | | | |
| | ITS2, intragenomic variability copy A, strain | | | |
| | A1 | | | |
| AY293968.1 | Coccomyxa chodatii internal transcribed | 559 | 92% | 90% |
| | spacer 1, 5.8S ribosomal RNA gene, and | | | |
| | internal transcribed spacer 2, complete | | | |
| | sequence | | | |
| FR850476.1 | Coccomyxa actinabiotis rRNA | 376 | 59% | 88% |
| | ITS1-5.8S-ITS2-28S | | | |
| AY328522.1 | Coccomyxa peltigerae strain SAG 216-5 | 366 | 31% | 94% |
| | 18S ribosomal RNA gene, partial sequence; | | | |
| | internal transcribed spacer 1, 5.8S ribosomal | | | |
| | RNA gene and internal transcribed spacer 2, | | | |
| | complete sequence; and 26S ribosomal | | | |
| | RNA gene, partial sequence | | | |
| HE586515.1 | Coccomyxa sp. KN-2011-T3 genomic DNA | 158 | 23% | 83% |
| | containing 18S rRNA gene, ITS1, 5.8S rRNA | | | |
| | gene and ITS2, strain T3 | | | |
| HE586550.1 | Coccomyxa sp. KN-2011-T1 genomic DNA | 158 | 23% | 83% |
| | containing 18S rRNA gene, ITS1, 5.8S rRNA | | | |
| | gene and ITS2, strain T1 | | | |
| HE586514.1 | Coccomyxa sp. KN-2011-T2 genomic DNA | 128 | 15% | 86% |
| | containing 18S rRNA gene, ITS1, 5.8S rRNA | | | |
| | gene and ITS2, strain T2 | | | |

This sequence comparison shows that the sequence of strain C-IR3-4C has 83% to 95% identity with the other Coccomyxae strains referenced, of the same order as the scores obtained for the comparison of the ITS region of the referenced Coccomyxae with one another (78% to 99%), and very far from that obtained for the comparison with other genera (about 30% to 50%).

It is thus a Coccomyxa species different from all those referenced heretofore. These results indicate, in fact, that the microalga isolated is a member of the genus Coccomyxa, but that its DNA differs sufficiently from that of the other species of Coccomyxae listed in the databases, in particular in terms of the ITS1 and ITS2 DNA, so as to be deemed a novel species, which is named herein Coccomyxa C-IR3-4C.

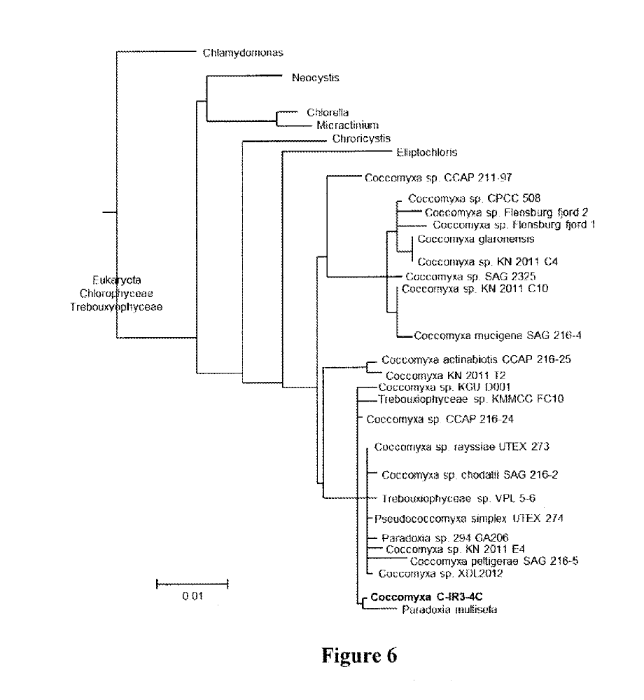

FIG. 6 presents the phylogenetic tree obtained after alignment of the DNA sequences corresponding to the 18S rRNA (BLASTN). The bar indicates a substitution rate of 1% (0.01).

### EXAMPLE 2

### Coccomyxa C-IR3-4C Resistance to Ionizing Radiation

The resistance to ionizing radiation of Coccomyxa C-IR3-4C algae was tested by exposing them to various doses of gamma radiation from decaying spent fuel. Post-irradiation mortality was determined by vital staining (neutral red).

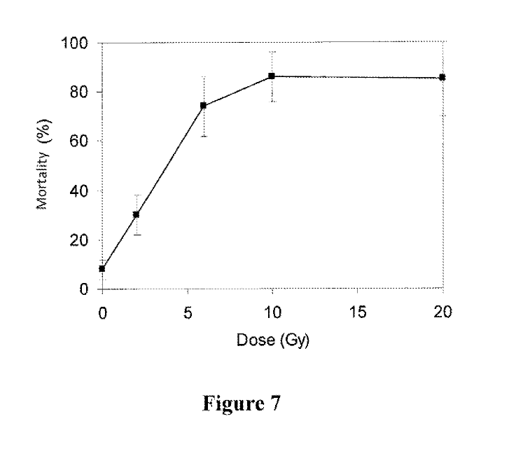

FIG. 7 shows the percentage of mortality of Coccomyxa C-IR3-4C as a function of irradiation dose, measured three days after acute irradiation. The ionizing radiation dose that kills half the population is between 2 and 6 kGy.

### EXAMPLE 3

### Decontamination of Water from Nuclear Plants by Means of Coccomyxa C-IR3-4C

Water with a composition typical of that of water from a spent fuel pool was brought into contact with Coccomyxae C-IR3-4C for 24 hours, under illumination. A fresh 100 mg mass of Coccomyxae C-IR3-4C algae first cultivated in liquid BBM and rinsed beforehand three times with Milli-Q water is brought into contact with 50 ml of water initially containing the beta-emitting radionuclides ³H (200,000 Bq/l) and ¹⁴C (1000 Bq/l) and the gamma-emitting radionuclides listed in Table IV below.

TABLE IV

| Radionuclide | ⁵⁴Mn | ⁶⁰Co | ^{110m}Ag | ¹³⁷Cs | ²³⁸U |
|---|---|---|---|---|---|
| Activity (Bq/l) | 9 | 42 | 23 | 67 | 21 |

The γ-spectrometry assay of the water and the algae 24 hours post-contact shows that 81% of the γ activity of the water is removed.

The percentage of γ-emitting radionuclides bound by the algae in 24 hours is indicated in Table V below.

TABLE V

| Radionuclide | ⁵⁴Mn | ⁶⁰Co | ^{110m}Ag | ¹³⁷Cs | ²³⁸U |
|---|---|---|---|---|---|
| Binding % | 52 | 45 | 100 | 95 | 100 |

All or virtually all of the ^{110m}Ag, the ¹³⁷Cs and the ²³⁸U and half of the ⁵⁴Mn and the ⁶⁰Co were purified from the water.

### EXAMPLE 4

### Decontamination of Storage Pool Water by Means of Coccomyxa C-IR3-4C Via In Situ Action

The Coccomyxae C-IR3-4C microalgae present in a spent nuclear fuel pool decontaminate the radionuclides present in said pool. Said pool is filled with water of pH between 6 and 7, of conductivity between 1 and 2 μS/cm and of mean temperature 25° C., and contains radioactive metal elements due to the materials stored therein. It is in contact with ambient air and is illuminated by neon lighting.

Under these conditions, Coccomyxa C-IR3-4C is able of colonize the storage racks within the pool and can live there and reproduce there for years.

Counts taken by γ spectrometry made it possible to determine the total activity and the nature and the activity of each γ emitter concentrated by about 1 g of fresh algal mass and are given in Table VI below. The activity of the microalgae is defined in this table as the ratio of the activity (Bq) of 1 g of fresh algal mass to the activity of 1 ml of the water in which the algae live.

TABLE VI

| | Activity of the microalgae/ |
|---|---|
| Radionuclide | Activity of the water |
| ⁶⁰Co | 67 |
| ⁶⁶Cu | 167 |
| ⁹²Sr | 7.5 |
| ¹¹⁰mAg | 62 |
| ¹³⁴Cs | 17 |
| ¹³⁷Cs | 8030 |
| Total | 8380 |

These results show that the algae are up to 10⁴ times more active than the water in which they live, and thus that they in fact concentrated the radioelements ⁶⁰Co, ⁶⁶Cu, ⁹²Sr, ^{110m}Ag, ¹³⁴Cs and ¹³⁷Cs.

The alga Coccomyxa C-IR3-4C has a cesium-137 (¹³⁷Cs) concentration factor, defined as the ratio of the concentration of Cs bound by the algae, in atoms/g of fresh matter, to the concentration of Cs in the water, in atoms/ml, of about 20,000.

### EXAMPLE 5

### Decontamination of Water from Nuclear Plants by Means of Coccomyxa chodatii

The determination of the ionizing-radiation resistance of Coccomyxa chodatii (strain SAG 216/2) algae shows that the ionizing radiation dose that kills half the population is about 1.5 kGy (Rivasseau et al., 2013, cited above). The ionizing-radiation resistance LD₅₀ of the algae is generally between 30 and 1200 Gy (IAEA, 1976, cited above).

It appears that the radiation tolerance of microalgae belonging to the genus Coccomyxa is higher than those of algae belonging to other genera, with exceptional resistance by Coccomyxa actinabiotis, in the same way that all bacteria belonging to the genus Deinococcus have high radiation tolerance.

In order to determine whether Coccomyxa chodatii shared the element uptake capacity of Coccomyxa actinabiotis and Coccomyxa C-IR3-4C, water with a composition typical of that of water from a spent nuclear fuel pool was brought into contact with Coccomyxae chodatii for 24 hours, under illumination. A fresh 110 mg mass of Coccomyxae chodatii algae first cultivated in liquid BBM and rinsed beforehand three times with Milli-Q water is brought into contact with 50 ml of water initially containing the beta-emitting radionuclides ³H (200,000 Bq/l) and ¹⁴C (1000 Bq/l) and the gamma-emitting radionuclides listed in Table IV above.

The assay of the water and the algae 24 hours post-contact shows that 80% of the γ activity of the water is removed.

The percentage of γ-emitting radionuclides bound by the algae in 24 hours is indicated in Table VII below.

TABLE VII

| Radionuclide | ⁵⁴Mn | ⁶⁰Co | ^{110m}Ag | ¹³⁷Cs | ²³⁸U |
|---|---|---|---|---|---|
| Binding % | 100 | 54 | 100 | 94 | 100 |

All or virtually all of the ^{110m}Ag, the ⁵⁴Mn, the ¹³⁷Cs and the ²³⁸U and half of the ⁶⁰Co were purified from the water.

### EXAMPLE 6

### Control of the Proliferation or the Removal of the Microalgae

Microalgae are photosynthetic. They need light to carry out photosynthesis and to produce their organic matter. In the various very low-nutrient media and effluents from nuclear power stations, their growth can thus be controlled via illumination. In order for them to grow at a given spot, it suffices to provide them with light. Their growth can also be controlled by providing them with light allowing little or no photosynthesis, for example with a yellow-green inactinic lamp.

The water can be filtered in order to capture the algae suspended therein, and thereby to control their growth.

A chemical method such as oxidation, for example with hydrogen peroxide, can be used to remove them completely. Five milliliters of 20 g/l hydrogen peroxide is added to medium containing 20 ml of microalgal cell suspension, i.e., a final H₂O₂ concentration of 4 g/l. After 1 day, the culture contains aggregates of brown/white matter and its green color has disappeared. At the end of 1 week, nothing can be observed under the microscope.

The breakdown of the algae by hydrogen peroxide is thus fast, gradual and total, leaving no organic matter residue.

This solution can also be used to clean radioactive parts transferred from one medium to another and to avoid any algal contamination of the new medium, or to clean the walls of empty pools.

### EXAMPLE 7

### Bioprocesses Employing Rare Earth Uptake by Coccomyxa CCAP 216/26

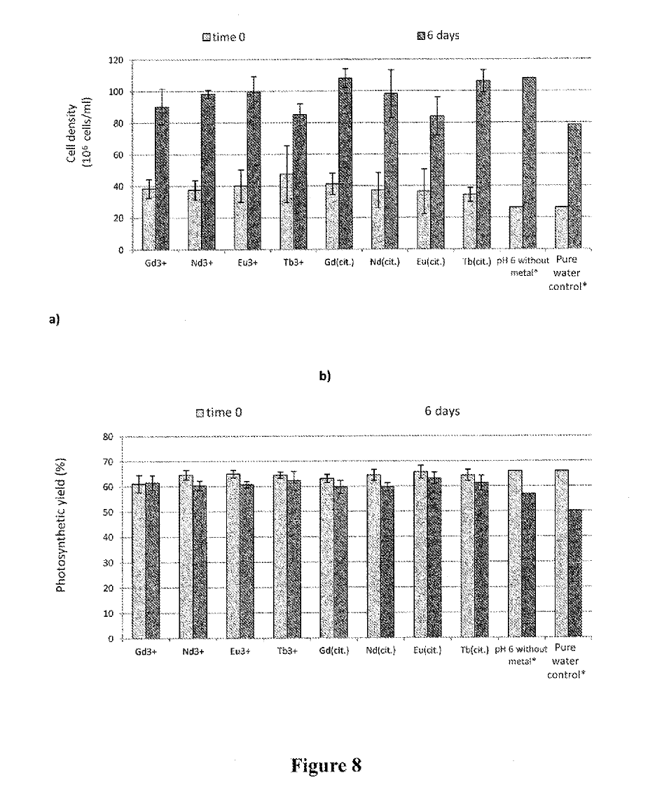

This example refers to FIGS. 8 to 13, the captions for which are presented below:

FIG. 8: Change in a) cell density and b) photosynthetic yield of Coccomyxa CCAP 216/26 microalgae exposed to 10⁻⁶ M rare earths at pH 6, optimal conditions for biological purification (n=3 biological replicates). Four rare earths (Gd, Nd, Eu and Tb) and two chemical forms (cation and citrate complexes) were tested in parallel. *=control samples not tested in parallel and not replicated. These values are presented as an indication of their relevance. The second sample was taken not after 6 days of exposure but after 5 days. The pH in pure water is 7±1.

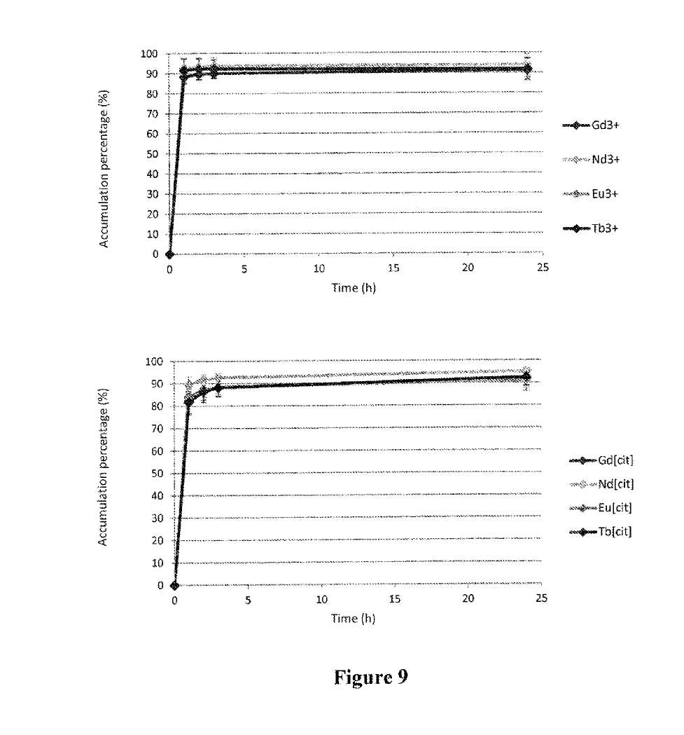

FIG. 9: Percentages of rare earths accumulated (%) by Coccomyxa CCAP 216/26 microalgae exposed to 10⁻⁶ M metal at pH 6, optimal conditions for biological purification (n=3 biological replicates). Four rare earths (Gd, Nd, Eu and Tb) and two chemical forms (cation and citrate complexes) were tested in parallel (mean±standard deviation).

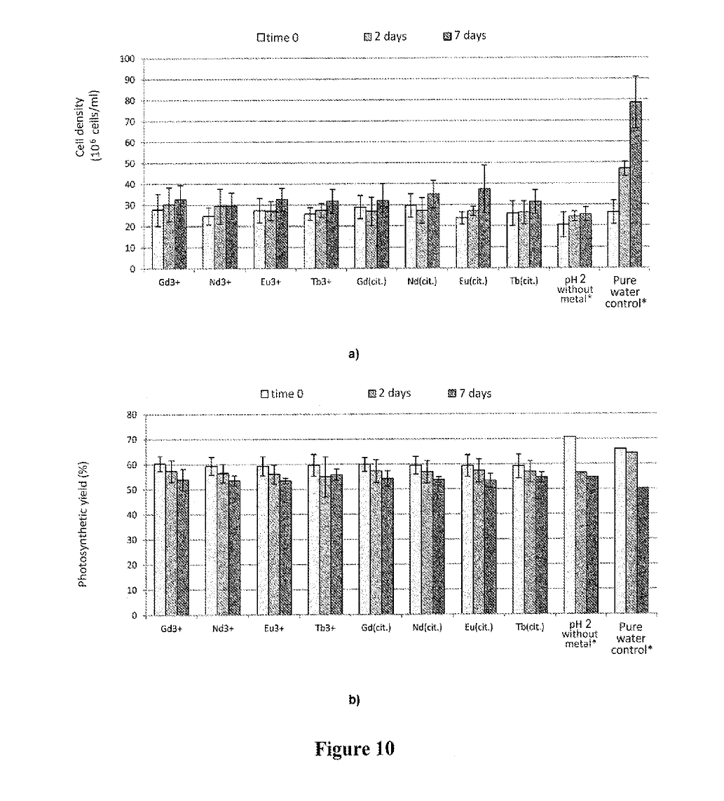

FIG. 10: Change in a) cell density and b) photosynthetic yield of Coccomyxa CCAP 216/26 microalgae exposed to 10⁻² M rare earths at pH 2, optimal conditions for metal uptake (n=5 biological replicates). Four rare earths (Gd, Nd, Eu and Tb) and two chemical forms (cation and citrate complexes) were tested in parallel. *=control samples not tested in parallel and not replicated. These values are presented as an indication of their relevance. The third sample was taken not after 7 days of exposure, but after 5 days. The pH in pure water is 7±1.

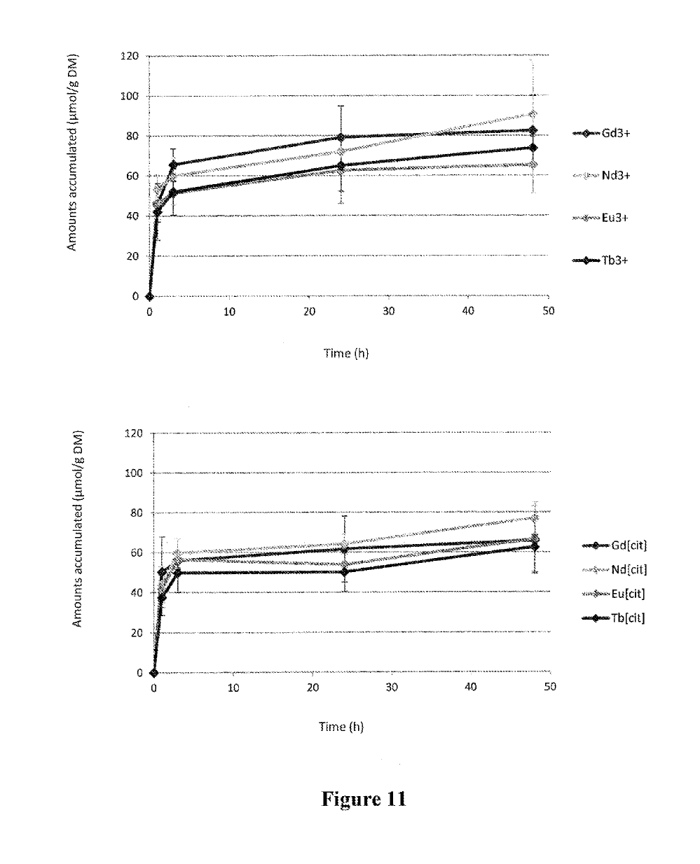

FIG. 11: Quantities of rare earths accumulated (μmol/g of dry matter) by Coccomyxa CCAP 216/26 microalgae exposed to 10⁻² M metal at pH 2, optimal conditions for metal uptake (n=3 biological replicates). Four rare earths (Gd, Nd, Eu and Tb) and two chemical forms (cation and citrate complexes) were tested in parallel.

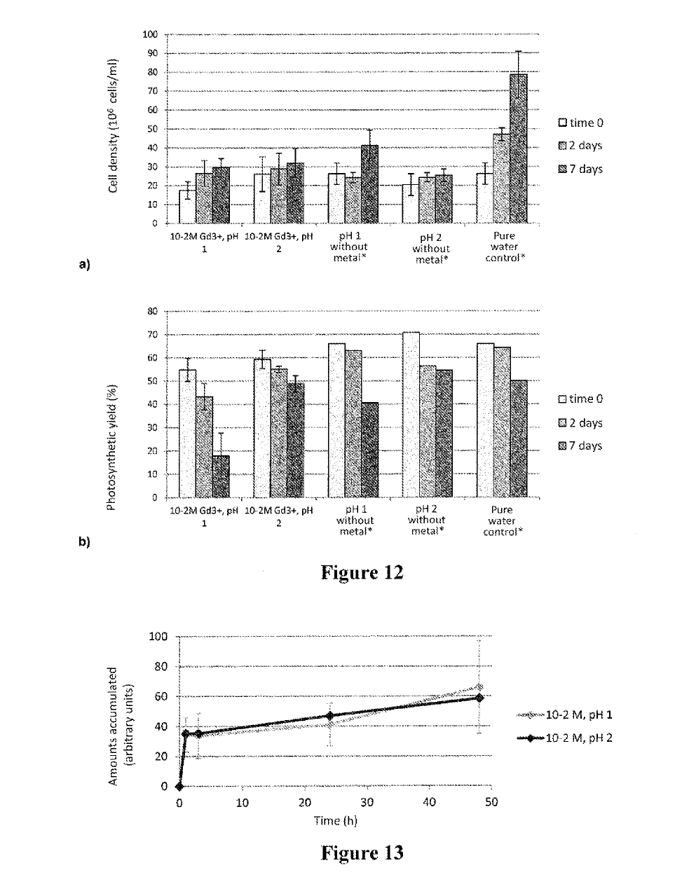

FIG. 12: Change in a) cell density and b) photosynthetic yield of Coccomyxa CCAP 216/26 microalgae exposed to 10⁻² M metal and at pH 1 or 2 (n=5 biological replicates). The two exposure conditions were tested in parallel. *=control samples not tested in parallel and not replicated. These values are presented as an indication of their relevance. The third sample was taken not after 7 days of exposure, but after 5 days. The pH in pure water is 7±1.

FIG. 13: Quantities of rare earths accumulated (arbitrary units) by Coccomyxa CCAP 216/26 microalgae exposed to 10⁻² M metal and pH 1 or 2 (n=3 biological replicates under the conditions 10⁻² M Gd³⁺ and pH 1; n=2 biological replicates under the conditions 10⁻² M Gd³⁺ and pH 2). The two exposure conditions were tested in parallel.

### 7.1. Experimental Protocols

7.1.1. Materials and Methods

In order to observe sterile conditions while culturing the algae, the protocol was carried out under a laminar flow hood, working within the sterile field created by a burner flame. The materials and solutions used were sterilized beforehand in an autoclave. The culture containers were covered with aluminum foil so as to allow gas exchange. On the other hand, the experiments in which the algae were exposed to metals were carried out under nonsterile conditions.

Centrifugations were carried out in a Megafuge 16R centrifuge (Thermo Scientific) equipped with a TX-400 rotor (Thermo Scientific). Suitable adapters (part nos. 75003683 and 75003682, Thermo Scientific) were used in order to centrifuge samples contained in 15 or 50 ml Falcon disposable centrifuge tubes (part nos. 734-0451 and 734-0448, VWR).

The microalgae were observed using an Optiphot light microscope (Nikon) equipped with ×20 and ×40 magnification objectives. Cell density was measured by counting 12 μl of algae suspension on an aluminum-coated Malassez counting chamber with 0.0025 mm² mini squares, 0.200 mm in depth (part no. 0640630, Marienfeld). The samples were diluted beforehand, if needed, so as to count between 50 and 100 cells per square.

Photosynthetic yield was measured in the dark using a PAM-103 chlorophyll fluorometer (Walz).

Rare earths were assayed in the liquids and in the algae by mass spectrometry coupled to a plasma torch, using the Hewlett-Packard 4500 ICP-MS System. The protocols used and the isotopes selected are described in paragraph 7.1.4.

7.1.2. Preparing the Algae

### Subculturing in Liquid Medium

The suspended algae were collected by centrifugation under gentle conditions (100 g, 25 min, 4° C., acceleration and deceleration 2). The algae contained in the pellet were then washed by adding sterile Milli-Q water followed by centrifugation, before being suspended in fresh sterile culture medium, with an initial cell density of 5·10⁶ cells/ml.

### Culturing the Algae

Coccomyxa CCAP 216/26 algae were cultivated in a 2-liter photobioreactor, in 1.5 liters of sterile culture medium consisting of BBM (B5282, Sigma-Aldrich) diluted 1:2 in Milli-Q water (0.5×). Temperature and luminosity were kept constant at 22±1° C. and 90±10 μmol/m²/s, respectively. The cells were agitated and aerated by means of continuous air bubbling. The initial concentration of the algae is about 5·10⁶ cells/ml. Fifteen milliliters of concentrated (50×) BBM was added every 3 to 4 days as of the second week of culture in order to nourish the cells. The photobioreactor was used in batch mode and was refreshed each month. The algae were used once the maximum cell density, greater than 100·10⁶ cells/ml, was reached (fed steady-state).

### Collecting and Washing the Algae

After collection by centrifugation (100 g, 25 min, 4° C., acceleration and deceleration 2), the algae were washed three times by adding sterile Milli-Q water followed by centrifugation (100 g, 10 min, 4° C., acceleration and deceleration 2) and removal of the supernatant. The washed algae were then taken up in sterile Milli-Q water and placed in an Erlenmeyer flask in an Infors incubator in which the temperature and the luminosity are kept constant (22±1° C. and 90±10 μmol/m²/s) and aeration is provided by shaking (100 rpm).

### Quantifying the Algae

Three 4 ml to 5 ml samples of the suspension of washed algae were filtered through pre-tared nitrocellulose filters of 1.2 μm pore size (part no. 512-0267, VWR). The filters were dried for 30 minutes at 70° C. and then weighed to determine an apparent concentration of dry matter. Measuring this value made it possible, by proportionality, to project the effective concentration of dry matter of the suspension and consequently to dilute the latter so as to obtain a concentration of 0.50±0.1 g/l of dry matter. The dry matter concentration of the suspension after dilution was controlled by drying the three 4 ml to 5 ml samples at 100° C. for 24 hours, after which the residue was weighed.

7.1.3. Algal Metal Uptake Experiments

### Exposing the Algae to Metals

For each experiment, 30 ml of the suspension of washed algae was placed in 100 ml Erlenmeyer flasks. The concentration of the algae is 0.5±0.1 g/l of dry matter. The pH was then adjusted to its set point by adding KOH or concentrated HCl. The samples were placed in an Infors incubator overnight in order for the algae to adapt to the pH. At t₀, 300 μl of LnCl₃ solution (Ln=the lanthanide studied), 100 times more concentrated than the desired final concentration, was added to the samples. The quantities added were verified by weighing. The pH was readjusted to its set point daily.

### Physiological Monitoring

The physiological state of the algae was monitored by measuring cell density and photosynthetic yield.

### Sampling the Supernatant

A 1.2 ml sample of the algae suspension was taken. The supernatant was separated from the algae by two successive centrifugations (100 g, 10 min, 4° C., acceleration and deceleration 3) (centrifugation, sampling the supernatant and then centrifugation of said supernatant). After diluting 1 ml of supernatant in 4 ml of 1.25% HNO₃ (each dilution being verified by weighing), the resulting solution was kept at 4° C. to await a subsequent assay of the metals in solution by ICP-MS.

### Sampling the Pellet

A 3 ml sample of the algae suspension was taken. The algae were collected by centrifugation (500 g, 10 min, 4° C., acceleration and deceleration 4). They were then washed once or twice by taking them up in 4 ml of Milli-Q water, centrifugation (500 g, 10 min, 4° C., acceleration and deceleration 4) and removal of the supernatant. The algae pellet was kept at 4° C. to await mineralization and an assay of the incorporated metals by ICP-MS.

7.1.4. Metals Assay

### Mineralization of the Algae

The algae were dry mineralized in 1.5 ml of aqua regia (65% HNO₃/30% HCl, 2:1 v/v) at 180° C. The residue was taken up in 1 ml of 10% HNO₃ (solution prepared by diluting commercially-available ultrapure 65% HNO₃ solution) and then diluted 1:10 with sterile Milli-Q water. Each dilution was verified by weighing.

### Dilution and Metals Assay

The solutions arising from the supernatant samples or from mineralization of the algae were diluted in 1% HNO₃ so as to obtain a metal concentration within the standard range. These samples were assayed by ICP-MS by comparison with a standard range prepared using solutions provided by Analab. Each dilution was verified by weighing. The metal concentration was calculated as the mean of the measurements taken on the various isotopes.

The rare-earth stock solution used for the algae exposure was also assayed.

For each element analyzed, the standard range and the isotopes measured are summarized in the Table.

TABLE VIII
ICP-MS analysis parameters

| Element | Standard range | Isotopes analyzed |
|---|---|---|
| Neodymium | 0.5 to 50 nM | ¹⁴²Nd, ¹⁴⁴Nd and ¹⁴⁶Nd |
| Europium | 0.5 to 10 nM | ¹⁵¹Eu and ¹⁵³Eu |
| Gadolinium | 0.1 to 10 nM | ¹⁵⁶Gd, ¹⁵⁸Gd and ¹⁶⁰Gd |
| Terbium | 0.2 to 20 nM | ¹⁵⁹Tb |

Ideally, the metals assay was carried out in the supernatants when the sample showed a high accumulation percentage and in the pellets when low accumulation percentages were observed.

7.1.5. Preparation of Ln[Citrate] Complexes

Citrate complexes were prepared by placing an equimolar amount of metal and citric acid in solution in Milli-Q water. According to the thermodynamic data, the complex then forms spontaneously.

### 7.2. Binding of Four Rare Earths Gadolinium, Neodymium, Europium and Terbium in Free Form and Complexed Form by Coccomyxa CCAP 216/26 Under Optimal Conditions for Biological Purification (10⁻⁶ M Metal, pH 6)

7.2.1. Experiments Performed

The accumulation of four rare earths (Gd, Nd, Eu and Tb) in two chemical forms (cation and citrate complex) by Coccomyxa CCAP 216/26 was tested under optimal conditions for accumulation percentage: 10⁻⁶ M rare earths, high pH. The pH conditions and the initial concentrations were optimized using central composite experimental designs, applied to a pH range between pH 2 and pH 9 and a concentration range between 10⁻²M and 10⁻⁶ M metal. The optima thus obtained, identical for two elements (Gd and Nd) and two chemical forms (cation and citrate complex), were generalized.

The experimental designs revealed an optimal pH of 6 for the cations and 9 for the citrate complex. However, the effect of pH on accumulation of the complex is very low and accumulations at pH 4, 6 or 8 are not significantly different. Consequently, during the present experiment, all the tests were performed at pH 6±1. 10⁻⁶ M rare earths.

The eight corresponding experiments were performed in parallel and were repeated three times over three weeks and with three different biomasses, in order to estimate intermediate reliability.

7.2.2. Physiological Monitoring

The physiological state of the algae was monitored by measuring photosynthetic yield and cell density (FIG. 8). The algae remain in excellent physiological state: cell density increases and photosynthetic yield remains close to 60%, even after 6 days of exposure to rare earths under the present conditions.

7.2.3. Monitoring the Accumulation of Metals

Accumulated metals were assayed in the supernatants in order to determine accumulation percentages (%). The values obtained are presented in Table IX and FIG. 9.
The accumulation percentages are of the same order of magnitude for all the rare earths: between 90% and 95%.
For a given element, there is no significant difference between the accumulation of the cation or the citrate complex after 24 hours in contact with the algae.
Virtually all of the rare earth cations accumulated by the algae are captured in less than 1 hour under the exposure conditions 10⁻⁶ M metal, pH 6. There is little or no variation thereafter, up to 24 hours of contact.
At least 3 hours of contact are necessary for accumulation of rare earths in citrate complex form under the conditions 10⁻⁶ M metal, pH 6. The accumulation percentage continues to increase slightly between 3 hours and 24 hours of contact.

TABLE IX
Percentages of rare earths accumulated (%) by Coccomyxa CCAP
216/26 microalgae exposed to 10⁻⁶M metal at pH 6, optimal
conditions for biological purification (n = 3 biological replicates)

| | Metal | 0 | 1 h | 2 h | 3 h | 24 h |
|---|---|---|---|---|---|---|
| Cation | Gd | 0.0 | 88.3 ± 3.3 | 89.8 ± 2.9 | 90.1 ± 2.6 | 91.2 ± 3.6 |
| | Nd | 0.0 | 93.0 ± 5.0 | 93.4 ± 4.7 | 93.8 ± 4.4 | 93.9 ± 5.3 |
| | Eu | 0.0 | 92.1 ± 5.0 | 92.3 ± 5.2 | 92.4 ± 4.7 | 91.8 ± 5.8 |
| | Tb | 0.0 | 91.6 ± 5.8 | 92.3 ± 5.3 | 92.3 ± 4.6 | 91.8 ± 5.3 |
| Citrate | Gd | 0.0 | 82.4 ± 6.1 | 86.6 ± 4.9 | 88.5 ± 4.2 | 91.9 ± 4.2 |
| complex | Nd | 0.0 | 89.7 ± 3.5 | 91.8 ± 2.3 | 92.7 ± 1.7 | 95.0 ± 0.5 |
| | Eu | 0.0 | 84.1 ± 3.6 | 87.3 ± 3.2 | 88.5 ± 3.2 | 91.0 ± 4.8 |
| | Tb | 0.0 | 81.7 ± 4.8 | 86.5 ± 3.8 | 88.2 ± 3.9 | 92.4 ± 3.8 |

### 7.3. Binding of Four Rare Earths Gadolinium, Neodymium, Europium and Terbium in Free Form and Complexed Form by Coccomyxa CCAP 216/26 Under Optimal Conditions for Metal Uptake (10⁻² M Metal, pH 2)

7.3.1. Experiments Performed

The accumulation of four rare earths (Gd, Nd, Eu and Tb) in two chemical forms (cation and citrate complex) by Coccomyxa CCAP 216/26 was tested under optimal conditions for the quantities accumulated: 10⁻² M rare earths, pH 2.

7.3.2. Physiological Monitoring

The physiological state of the algae was monitored by measuring photosynthetic yield and cell density (FIG. 10). Growth of the algae exposed to pH 2, in the presence or absence of rare earths, stops completely. Photosynthetic yield decreases slightly after 6 days of exposure to 10⁻² M rare earths at pH 2. It remains above 50% after 2 days of exposure, which testifies to the good viability of the algae during the first 48 hours.

7.3.3. Monitoring the Accumulation of Metals

Metals were assayed in the algae pellets after mineralization with aqua regia in order to determine the quantities of rare earths accumulated (μmol/g of dry matter). The values obtained are presented in Table X and FIG. 11.

On the whole, the quantities of rare earths accumulated are relatively similar. Whatever the element or chemical form studied, the amount of metal accumulated is comprised between 40 and 100 μmol/g of dry matter after a contact time of 3 hours or 24 hours. Small differences are observed in the details.
Under the exposure conditions 10⁻² M metal at pH 2, there is a significant difference in accumulation between the various chemical elements. Gadolinium and neodymium appear to be more easily captured than europium and terbium (101 and 78 μmol/g of metal dry matter captured in 24 hours for Gd³⁺ and Nd³⁺, respectively; 43 and 39 μmol/g of metal dry matter captured in 24 hours for Eu³⁺ and Tb³⁺, respectively).
In the case of gadolinium and neodymium, accumulation of the cations appears to be slightly more efficient than that of the citrate complexes. The cation accumulation maxima are reached within 24 hours of contact. Whatever the state of the biomass, repeatability is good up to 24 hours (FIG. 11). At 48 hours of contact, toxicity can begin to set in depending on the state of the biomass. Citrate complexes accumulate quickly during the first 3 hours of contact, then the increase is more gradual up to 48 hours of exposure.
In the case of europium and terbium, the difference in accumulation between the two chemical forms does not appear to be significantly different. The large majority of the metals captured by the algae are captured within only 3 hours. Little variation is visible thereafter.

TABLE X
Quantities of rare earths accumulated (μmol/g of dry matter) by
Coccomyxa CCAP 216/26 microalgae exposed to 10⁻²M metal at pH 2, optimal
conditions for metal uptake (n = 3 biological replicates).

| | Metal | 0 | 1 h | 3 h | 24 h | 48 h |
|---|---|---|---|---|---|---|
| Cation | Gd | 0.0 | 45.6 ± 8.6 | 65.6 ± 8.0 | 79.1 ± 15.9 | 82.4 ± 17.7 |
| | Nd | 0.0 | 53.2 ± 7.4 | 60.0 ± 9.2 | 72.0 ± 7.5 | 90.7 ± 26.9 |
| | Eu | 0.0 | 46.6 ± 8.3 | 51.4 ± 5.7 | 62.5 ± 16.4 | 65.3 ± 14.2 |
| | Tb | 0.0 | 42.1 ± 14.1 | 52.0 ± 11.6 | 65.0 ± 12.8 | 73.7 ± 8.8 |
| Citrate complex | Gd | 0.0 | 50.4 ± 17.6 | 56.0 ± 4.2 | 61.6 ± 16.5 | 65.9 ± 2.6 |
| | Nd | 0.0 | 44.1 ± 9.0 | 59.8 ± 7.1 | 64.2 ± 5.0 | 77.0 ± 8.1 |
| | Eu | 0.0 | 42.0 ± 6.8 | 56.5 ± 4.1 | 53.8 ± 1.2 | 66.5 ± 16.5 |
| | Tb | 0.0 | 37.5 ± 8.7 | 49.9 ± 9.7 | 50.1 ± 9.6 | 62.6 ± 13.3 |

The use of the cationic form of the rare earths, more quickly captured by the algae in the case of certain elements and less expensive in terms of reagents, thus seems preferable under the optimal conditions for recovering metals.

### 7.4. Binding of the Gd³⁺ Ion by Coccomyxa CCAP 216/26 Under Lower pH Conditions (pH 1)

7.4.1. Experiments Performed

In order to test the binding of rare earths under conditions as similar as possible to the solutions used to dissolve urban waste, the accumulation of Gd³⁺ by Coccomyxa CCAP 216/26 was tested under pH conditions lower (pH 1) than those tested heretofore:

10⁻² M Gd³⁺ and pH 2

10⁻² M Gd³⁺ and pH 1

The two experiments were performed in parallel.

7.4.2. Physiological Monitoring

The physiological state of the algae was monitored by measuring photosynthetic yield and cell density (FIG. 12). Growth of the algae exposed to pH 1 and 2, in the presence and in the absence of rare earths, slows significantly. Photosynthetic yield is affected by the exposure conditions tested here, but the algae are still viable at 48 hours and 6 days during exposures to 10⁻² M Gd³⁺, pH 1. After 6 days of exposure, the photosynthetic yield of the algae exposed to 10⁻² M Gd³⁺ and pH 2 remain close to 50%. The photosynthetic yield of algae exposed to 10⁻² M and pH 1 falls below 20% in 6 days.

7.4.3. Monitoring the Accumulation of Metals

The metals were assayed in the algae pellets after mineralization with aqua regia in order to determine the quantities of rare earths accumulated (μmol/g of dry matter). The values obtained are presented in Table XI and FIG. 12.

The algae are able to accumulate Gd³⁺ even when they are exposed for 48 hours to very low pH (pH 1). The accumulated quantities are higher at pH 2 than at pH 1.

The highest accumulation is observed after the algae are exposed for 24 hours to 10⁻²M Gd³⁺ and pH 2. An amount close to 100 μmol/g of dry matter is then captured by the algae. This condition remains the optimal condition for metal uptake.

TABLE XI
Quantities of rare earths accumulated (arbitrary units) by
Coccomyxa CCAP 216/26 microalgae exposed to 10⁻²M metal and pH 1 or 2
(n = 3 biological replicates under the conditions 10⁻²M Gd³⁺ and
pH 1; n = 2 biological replicates under the conditions 10⁻²M Gd³⁺ and pH 2).

| Concentration | pH | 0 | 1 h | 3 h | 24 h | 48 h |
|---|---|---|---|---|---|---|
| 10⁻²M | 1 | 0.0 | 34.6 ± 11.6 | 33.8 ± 15.1 | 41.2 ± 14.2 | 65.9 ± 31.0 |
| 10⁻²M | 2 | 0.0 | 35.1 ± 2.1 | 35.4 ± 1.0 | 47.0 ± 0.4 | 58.6 ± 0.2 |

1. A unicellular green alga of the genus Coccomyxa, comprising in the 18S ribosomal RNA-ITS1-5.8S ribosomal RNA-ITS2-28S ribosomal RNA genes of a sequence having at least 96% identity with the sequence of SEQ ID NO: 1.

2. The unicellular green alga of claim 1, having the features of the Coccomyxa strain deposited on 29 Jan. 2013 with the Culture Collection of Algae and Protozoa (CCAP) under number CCAP 216/26.

3. The unicellular green alga of claim 1, wherein the unicellular green alga is the Coccomyxa strain deposited on 29 Jan. 2013 with the CCAP under number CCAP 216/26.

4. A method of capturing at least one radioactive or nonradioactive element selected from the group consisting of Cs, Ag, Co, Mn, Sr, Cu, Cr, Zn, Ni, Fe, Sb, U, actinides, lanthanides and rare earths, such as Sc, Y, La, Ce, Pr, Nd, Pm, Sm, Eu, Gd, Tb, Dy, Ho, Er, Tm, Yb, Lu, and/or at least one of the radioisotopes ¹⁴C and ³H, from an aqueous medium containing said element and/or said radioisotope in solution, comprising incubating, in said aqueous medium, unicellular green algae of the genus Coccomyxa selected from Coccomyxa chodatii and the green alga of claim 1.

5. A method of capturing at least one radioactive or nonradioactive element selected from the group consisting of Sr and Cu, from an aqueous medium containing said element in solution, comprising incubating, in said aqueous medium, unicellular green algae of the genus Coccomyxa.

6. The method of claim 4, wherein said aqueous medium is radioactive medium.

7. The method of claim 4, wherein said aqueous medium is nonradioactive medium.

8. The method of claim 6, for capturing a metal selected from the group consisting of Ag, Co, Cs, U, Mn, Cu and Sr, wherein said metal is in the form of a radioactive isotope, or in the form of a mixture of isotopes.

9. The method of claim 4, wherein said green algae are combined with at least one other microorganism and/or at least one multicellular plant.

10. The method of claim 4, wherein the growth of the green algae of the species Coccomyxa C-IR3-4C and/or the species Coccomyxa chodatii is controlled by regulating the illumination of said aqueous medium.

11. The method of claim 4, further comprising recovering said element from the algae, wherein the radioactive or nonradioactive element captured is selected from the group consisting of Cs, Ag, Co, Mn, Sr, Cu, Cr, Zn, Ni, Fe, Sb, actinides, lanthanides and rare earths, such as Sc, Y, La, Ce, Pr, Nd, Pm, Sm, Eu, Gd, Tb, Dy, Ho, Er, Tm, Yb, Lu.

12. The method of claim 4, wherein the pH of the aqueous medium is between 1 and 6.

13. A method of depolluting an aqueous medium containing at least one radioactive or nonradioactive element selected from the group consisting of Cs, Ag, Co, Mn, Sr, Cu, Cr, Zn, Ni, Fe, Sb, actinides, lanthanides and rare earths, and/or at least one of the radioisotopes ¹⁴C and ³H, comprising incubating the aqueous medium with a green alga of the genus Coccomyxa being selected from Coccomyxa chodatii and the green alga of claim 1.

14. The method of claim 13, wherein said medium is radioactive.

15. A method of decontaminating an aqueous medium containing at least one radioactive or nonradioactive element selected from the group consisting of Sr and Cu, comprising incubating the aqueous medium with a green alga of the genus Coccomyxa.

16. The method of claim 14, wherein said green alga is combined with at least one other radioresistant or radiotolerant microorganism and/or at least one radioresistant or radiotolerant multicellular plant.

17. The method of claim 15, wherein said green alga is combined with at least one other radioresistant or radiotolerant microorganism and/or at least one radioresistant or radiotolerant multicellular plant.


SEQUENCE LISTING (3 sequences)

SEQ ID NO: 1 - DNA, 3101 bp, Artificial Sequence
  Coccomyxa C-IR3-4C alga
tcgacaatctggttgatcctgccagtagtcatatgcttgtctcaaagattaagccatgcatgtctaagtataaactgctttatactgtgaaactgcgaatggctcattaaatcagttatagtttatttgatggtaccttactactcggataaccgtagtaattctagagctaatacgtgcggaaatcccgacttctggaagggacgtatttattagataaaaggccgaccgggcttgcccgaaacgcggtgaatcatgataactccacgaatcgcatggcctcagcgccggcgatgtttcattcaaatttctgccctatcaactttcgacggtaaggtattggcttaccgtggtggtaacgggtgacggaggattagggttcgattccggagagggagcctgagaaacggctaccacatccaaggaaggcagcaggcgcgcaaattacccaatcttgacacaaggaggtagtgacaataaataacaataccggggtttttcaactctggtaattggaatgagtacaatctaaaccccttaacgaggatcaattggagggcaagtctggtgccagcagccgcggtaattccagctccaatagcgtatatttaagttgttgcagttaaaaagctcgtagttggatttcgggcgggcccggccggtccgcctttgggtgtgcactgaccgggcccgtcttgttgccggggacgggctcctgggcttaactgtccgggactcggagtcggcgaggttactttgagtaaattagagtgttcaaagcaggcctacgctctgaatacattagcatggaataacacgataggactctggcctatcttgttggtctgtgggaccggagtaatgattaagagggacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcctagtctcaaccataaacgatgccgactagggattggcgggcgttctgttgatgaccccgccagcaccttatgagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagcctgctaactagtcacgattggttcttccagtcggccgacttcttagagggactattggcgactagccaatggaagtgtgaggcaataacaggtctgtgatgcccttagatgttctgggccgcacgcgcgctacactgatgcaatcaacgagcctagccttggccgacaggtccgggtaatctttgaaactgcatcgtgatggggatagatgattgcaattattcatcttcaacgaggaatgcctagtaagcgcgagtcatcagctcgcgttgattacgtccctgccctttgtacacaccgcccgtcgctcctaccgattgggtgtgctggtgaagcgttcggattggcggcagtgcgcggttcgccgctcgctgcagccgagaagttcgttaaaccctcccacctagaggaaggagaagtcgtaacaaggtttccgtaggtgaacctgcggaaggatcattgcatcgatcgaacacaaaccgcgaacccacgtatccccgcgggcggctcgtctttctttccgaataaagagagccggccgtgcgcttgtccctgtccggttccgcttcggcggaacgggcgggtctggcgggctggggaggccgctctccctttaccgggaaagggccgaaccccggctggcaaaaccccattcgatccgaaaccagaaaccacctgaagatccggacggctgttggacagccgtctccaaccgaagacaactctcaacaacggatatcttggctcccgtatcgatgaagaacgcagcgaaatgcgatacgtagtgtgaattgcagataatgtgaatcatcgaatctttgaacgcaaattgcactcgaggctccggccgagagtatgtctgcctcagcgtcggcttcaccctcacccctctccatcgagaggcgtggacctggccgtcccggttaatgcaaccgggtctgccgaagaacagaggctcgaacgaggacccattcgggacgacagtctggtaggtgacccgtcagggatcgcatgccgctgtcggcccgggacctcgttgtaagccggcaggaatcggcgttcgcttctttgtaaaaagagtagcggccgcgcgagacacccctttcgacctgagttcaggcaagaacacccgctgaacttaagcatatcaataagcggaggaaaagaaactaaccaggattcccctagtagcggcgagcgaaccgggataagcccaacttgaaaatctccgtccttcgggcggcgaattgtagtctagagaggtgttctctgcggcgggccgtgcccaagtcccttggaagggggcgtccgagagggtgataaccccgtcggccacggtctcagccgctccacgaggcgctttcgcagagtcgggttgtttgggaatgcagcccaaagcaggtggtaagtcccatctaaggctaaatactgacgggagaccgatagcgaacaagtaccgcgagggaaagatgaaaagaactttgaaaagagagttaaaaagtgcttgaaattgttgggagggaagggattggaggtcgtgggtgcgcccaggctcacgccggcctcacggccggctgtatgtgctgggcgctggtcagcatcggttagtcgggcgggacaacagcggcctttgatcggccgcccctgtcgcccggctgtccgaggaatgaagggcgctctcggccttcgggacctgcgctctcaggatgctggcggaaggcctccaatctgcccgtcttgaaacacggaccaaggag

SEQ ID NO: 2 - DNA, 25 bp, Artificial Sequence
  primer
tcgacaatctggttgatcctgccag

SEQ ID NO: 3 - DNA, 26 bp, Artificial Sequence
  primer
ctccttggtccgtgtttcaagacggg